(19) United States
(12) Patent Application Publication
(10) Pub. No.: US 2011/0300022 A1
(43) Pub. Date: Dec. 8, 2011

(54) TEMPERATURE-ADJUSTABLE BIOCHIP ASSEMBLY

(76) Inventors: Chih-Hui Yang, Chiayi City (TW); Keng-Shiang Huang, Chiayi County (TW); Hsiang-Chen Hsu, Taipei City (TW)

(21) Appl. No.: 12/794,331

(22) Filed: Jun. 4, 2010

(51) Int. Cl. G01N 33/00

(52) U.S. Cl. 422/68.1

(57) ABSTRACT

A temperature-adjustable biochip assembly has a biochip with a microfluidic channel embedded inside and a temperature-controlling unit including a substance sited at a surface of the biochip, a cyclic guiding module passing through the substance, and a fluid flowed inside the cyclic guiding module, with the cyclic guiding module having a fluidic installed in the substance for guiding the fluid and a temperature-controlling component for modulating the temperature of fluid.

### BACKGROUND OF THE INVENTION

1. Field of the Invention

The present invention relates to a biochip assembly, particularly to a temperature-adjustable biochip assembly.

2. Description of the Related Art

A conventional temperature-adjustable biochip assembly as disclosed in Taiwanese Patent Nos. 451052, I310890, I230257 and I276601 comprises a biochip and a polar layer, wherein a micropipe is embedded in the biochip for fitting liquids flowed inside. Moreover, the polar layer is placed on the biochip and adjacent to the micropipe.

The polar layer of the conventional temperature-adjustable biochip assembly as described above is made from a metal, which can provide thermal energy via electric approach to heat the liquids flowed inside the micropipe.

However, the polar layer is performed like an electric resistance which can only transfer the electricity into thermal energy to heat the biochip but not further cool down the biochip after heating. Additionally, the temperature-controlling of the polar layer is not precise and stable, so that the utility of the conventional temperature-adjustable biochip assembly is limited.

Furthermore, due to the complexity of the manufacturing process for installing the polar layer on a biochip, the price of the conventional temperature-adjustable biochip assembly is usually high. Also the polar layer is irremovably inlayed in the biochip so as to bring more inconvenience and disadvantages in use.

Other conventional temperature-adjustable biochip assemblies as disclosed in Taiwanese Patent Nos. I247892, I272242 and M274370 comprise a biochip and a heater, wherein a microfluidic channel is disposed on the biochip to fit liquids flowed inside, with the heater to heat up the flowing liquid in the microfluidic channel.

However, the heater of the conventional temperature-adjustable biochip assemblies described in Taiwan Patent Nos. I247892, I272242 and M274370 is a polar heater which may have the same disadvantages, i.e., a complex process, non-disassembly, and unstableness of controlling-temperature as is mentioned above.

As a result, there is a need of improving the quality of the conventional temperature-adjustable biochip assembly.

### SUMMARY OF THE INVENTION

The primary objective of this invention is to provide a temperature-adjustable biochip assembly, which can control the temperature of a biochip well.

The secondary objective of this invention is to provide a temperature-adjustable biochip assembly, which contains a temperature-controlling unit for rapid, steady heating or cooling of the biochip.

Another objective of this invention is to provide a temperature-adjustable biochip assembly, which can improve the stability of temperature-controlling.

Another objective of this invention is to provide a temperature-adjustable biochip assembly, so that the manufacture of a biochip is simplified.

Another objective of this invention is to provide a temperature-adjustable biochip assembly, which can be easily disassembled.

A temperature-adjustable biochip assembly comprises a biochip with a microfluidic channel embedded inside and a temperature-controlling unit including a substance sited at a surface of the biochip, a cyclic guiding module passing through the substance, and a fluid flowing inside the cyclic guiding module, with the cyclic guiding module having a microfluidic channel installed in the substance for guiding the fluid and a temperature-controlling component for modulating the temperature of the fluid.

Further scope of the applicability of the present invention will become apparent from the detailed description given hereinafter. However, it should be understood that the detailed description and specific examples, while indicating preferable embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings, which are given by way of illustration only and thus are not limitative of the present invention, and wherein:

FIG. 1 is an exploded diagram illustrating a temperature-adjustable biochip assembly of the present invention;

FIG. 2 is another exploded diagram illustrating a temperature-adjustable biochip assembly and substance of the present invention;

FIG. 3 is a top view of a temperature-adjustable biochip assembly of the present invention;

FIG. 4 is a cross-sectional view of a temperature-adjustable biochip assembly of the present invention;

FIG. 5 is a line chart illustrating the relationship of temperature between a fluid and a biochip in the present invention;

FIG. 6 is a diagram illustrating the stability of temperature-control of a temperature-adjustable biochip assembly of the present invention;

In the various figures of the drawings, the same numerals designate the same or similar parts.

### DETAILED DESCRIPTION OF THE INVENTION

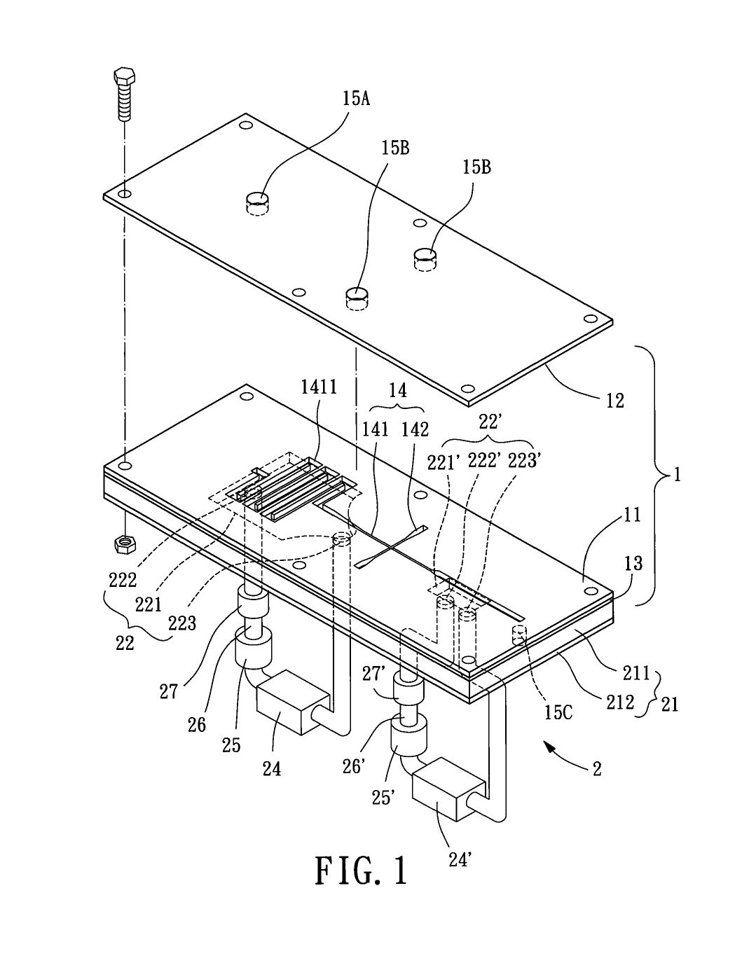

As shown in FIG. 1, a temperature-adjustable biochip assembly in the present invention comprises a biochip 1 and a temperature-controlling unit 2, wherein the temperature-controlling unit 2 is installed on a surface of the biochip 1.

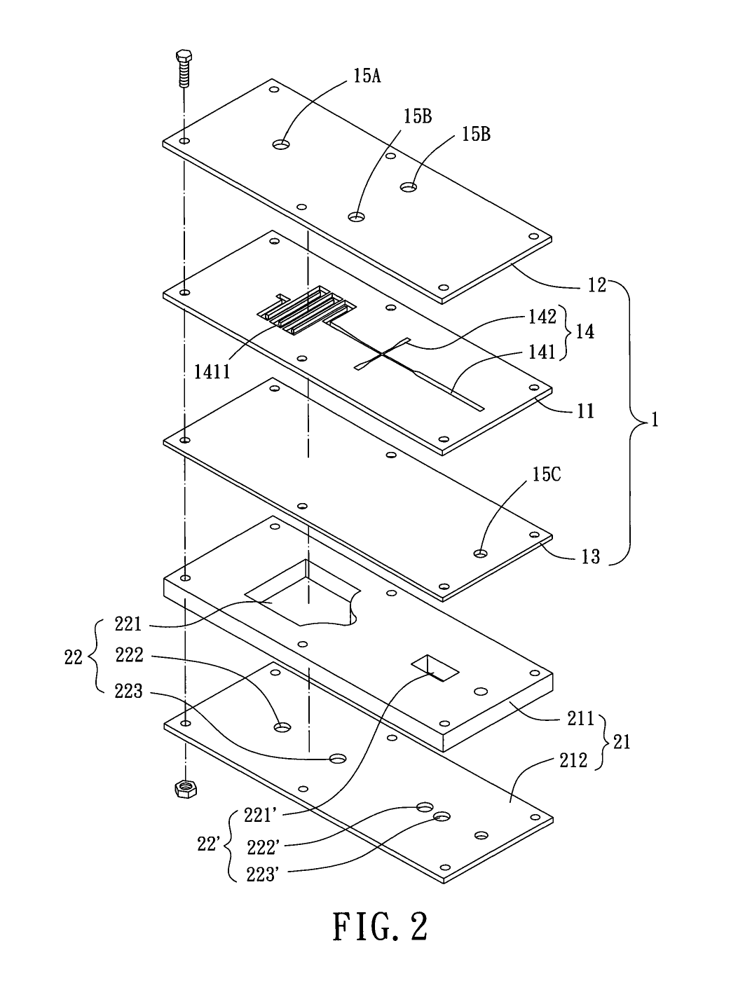

Referring to FIGS. 1 and 2, the biochip 1 can be made from glass, polymer and acrylic. As an example, the biochip 1 further comprises a first plate 11, a second plate 12, a third plate 13, at least one microfluidic channel 14 and a plurality of binding holes 15A, 15B and 15C, wherein the second plate 12 and the third plate 13 are differentially placed on two opposite faces of the first plate 11. In the present invention, a strategy to conjugate the first plate 11, the second plate 12 and the third plate 13 can be selected from a group of screw binding, thermal binding and adhesive binding, while screw binding is used as the example. The microfluidic channel 14 is embedded in the first plate 11, which consists of a main microfluidic channel 141 and a sub microfluidic channel 142 crossing through each other. The main microfluidic channel 141 contains a winding portion 1411 in order to extend the distribution of the main microfluidic channel 141. The binding hole 15A and 15 B are sited on the second plate 12 extending through one side of the second plate 12 to the other side thereof, and the binding hole 15C is sited on the third plate 13 extending through one side of the third plate 13 to the other side thereof. The binding holes 15A, 15B and 15C are connected with the front, middle and back of the microfluidic channel 14 individually.

Referring to FIGS. 1, 2 and 3, the temperature-controlling unit 2 can also be conjugated to the biochip 1 via screw binding, thermal binding or adhesive binding. The connection is preferably performed by screw binding, which makes the temperature-controlling unit 2 easily removable from the biochip 1. The temperature-controlling unit 2 comprises a substance 21, a fluidic 22, a fluid 23 and a temperature-controlling component 24. The substance 21 is placed on the other side of the third plate 13, with the binding hole 15C extending through one side of the substance 21 to the other side. In the example, the substance 21 is stacked up with a first board 211 and a second board 212, wherein the first board 211 is placed between the third plate 13 and the second board 212. The fluidic 22 is embedded in the substance 21, which includes a fluid tank 221, an inlet 222 and an outlet 223. The fluid tank 221 is installed on a face of the first board 211 facing the third plate 13. The inlet 222 and the outlet 223 are arranged to extend through the second board 212 and communicate with the fluid tank 221. Besides, the temperature-controlling unit 2 further includes a fluid 23, a temperature-controlling component 24, a driver 25, a delivering channel 26, and a fluid-controlling valve 27, while the fluidic 22, the temperature-controlling component 24, the driver 25, the delivering channel 26 and a fluid-controlling valve 27 jointly construct a cyclic guiding module. The fluid 23 is driven by the driver 25 to cyclically flow in other elements of the temperature-controlling unit 2. The temperature-controlling component 24, driver 25, and fluid-controlling valve 27 are serially connected by the delivering channel 26 for connecting the inlet 222 and outlet 223. The fluid 23 can be water, acetone or other liquids, preferably water, for being heated or cooled by the temperature-controlling component 24. Therefore, the temperature of fluid 23 is steadily heated, cooled or maintained by the temperature-controlling component 24. Meanwhile, the driver 25, such as a pump, admitting the running of the fluid 23 from the inlet 222 to the fluid tank 221, outlet 223, temperature-controlling component 24, driver 25, fluid-controlling valve 27, and back to the inlet 222 sequentially through the delivering channel 26. Moreover, preferably, there is another set of the combination of a fluidic 22′, a fluid 23′, a temperature-controlling component 24′, a driver 25′, a delivering channel 26′, and a fluid-controlling valve 27′, which are coupled and cooperated in the same way with that of the provided ones. Accordingly, the temperature-controlling components 24, 24′ can respectively maintain the fluids 23, 23′ at different temperatures so that the temperature-controlling unit 2 can provide heating and cooling functions at the same time. Thus, according to the placement of the temperature-adjustable biochip assembly summarized above, the manufacture of the temperature-controlling unit 2 can be easily achieved. Therefore, complex manufacturing processes of the polar layer will no longer be needed. Additionally, in the present invention, the temperature of the biochip 1 is under the control of the fluids 23, 23′ which can efficiently and precisely control or maintain the temperature of biochip 1, whether for heating up the temperature or cooling down the temperature until it is under room temperature. As a result, the temperature-adjustable biochip assembly in the present invention is beneficial to simplify the manufacturing process, the disassembling process, and temperature-controlling of the biochip.

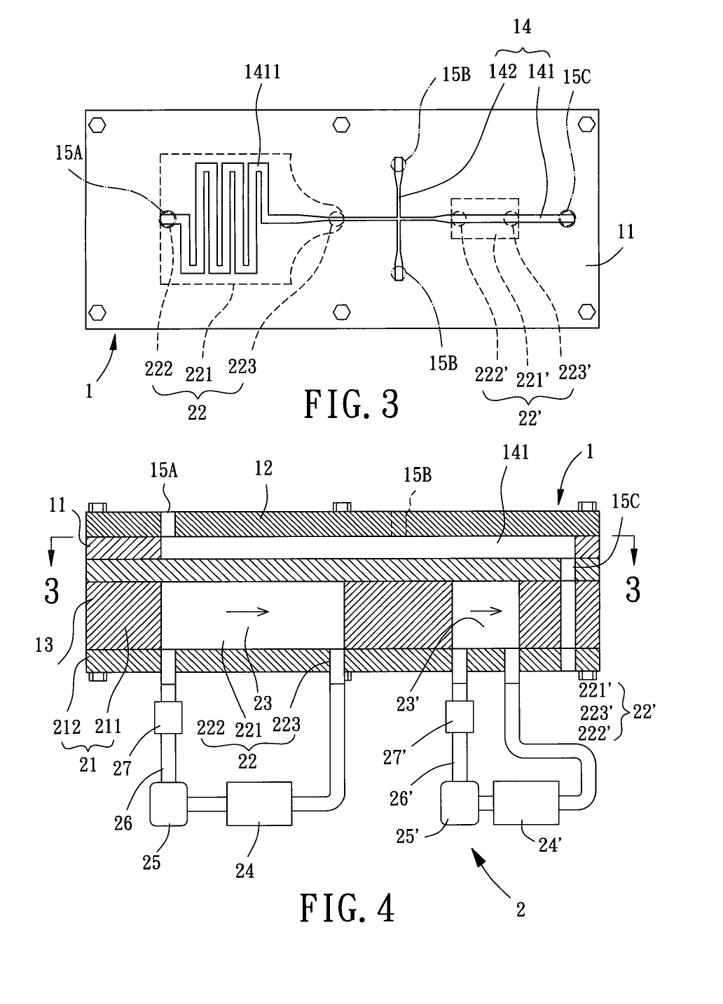

Referring to FIG. 3, the temperature-adjustable biochip assembly in the present invention is applied to manufacture into a condensed drug carrier. As an example, two temperature-controlling components 24, 24′ are installed and differentially connected to two fluidics 22, 22′ and two drivers 25, 25′ by two delivering channels 26, 26′ for heating or cooling fluids 23, 23′ individually by two temperature-controlling components 24, 24′. On the other hand, the driving and flow of the fluids 23, 23′ are propelled by the drivers 25, 25′ and the fluid-controlling valves 27, 27′, for admitting the running of the fluids 23, 23′ running from the inlets 222, 222′ through the fluidics 22, 22′ to the fluid tanks 221, 221′ in order to heat up or cool down the microfluidic channel 14. Furthermore, due to the location of the fluid tanks 221, 221′ (right below the microfluidic channel 14), the fluids 23, 23′ flowed in the microfluidic channel 14 can be directly and widely heated or cooled by the temperature-controlling components 24, 24′. For manufacturing into a condensed drug carrier, a condensed solution is filled in the binding hole 15A and further heated by the fluid tank 221 to promote its motility. Meanwhile an oil is filled in the binding hole 15B in order to shape the condensed solution into particles under the reaction of oil-water immiscibility as it runs through the middle of the microfluidic channel 14. In this way, the particles of condensed solution can be cooled down by the fluid tank 221′ to obtain several granules for further application to the pharmaceutical industry as drug carriers. Therefore, it is suggested that the temperature-adjustable biochip assembly of the present invention is useful to manufacture into drug carriers.

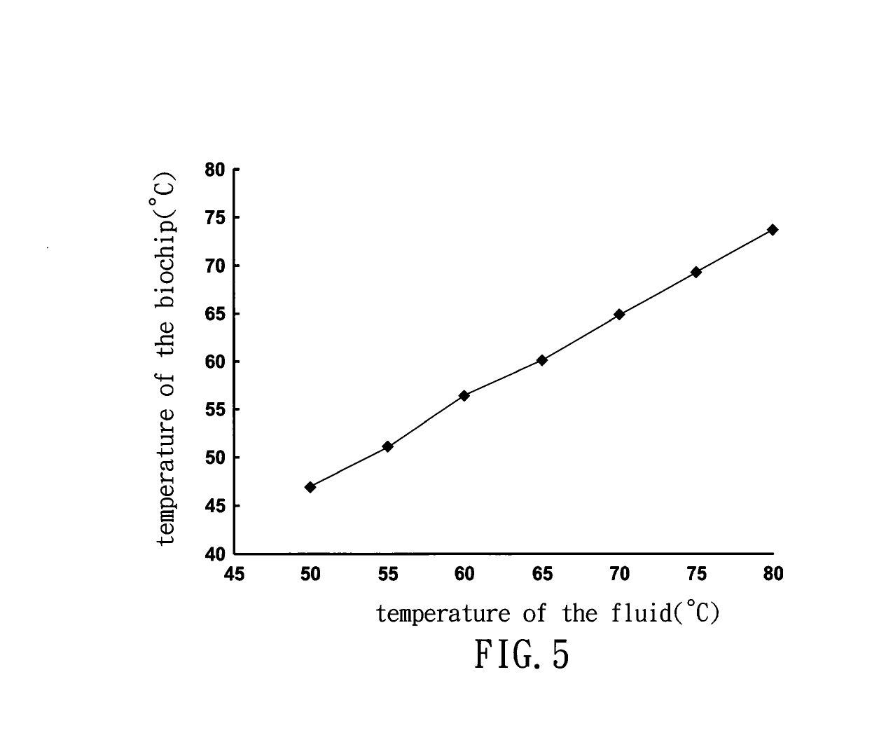

Referring to FIG. 5, this figure summarizes the relationship between the temperature of water (as fluids 23, 23′ in the example) and the temperature of biochip 1, wherein the difference in temperature between the water and the biochip 1 is significantly small. Hence, it is suggested that the temperature-adjustable biochip assembly is efficient in temperature control of the biochip.

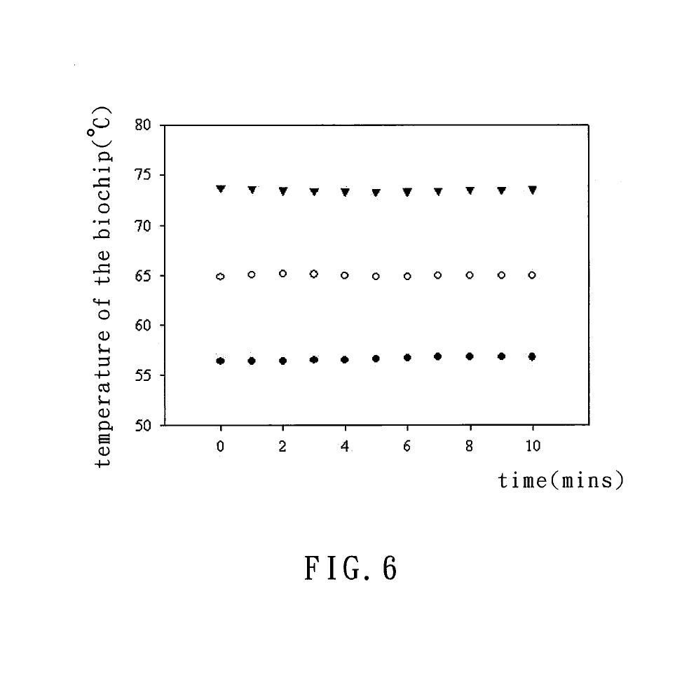

Referring to FIG. 6, this figure summarizes maintenance of the temperature-adjustable biochip assembly, wherein temperature-control of the temperature-adjustable biochip assembly is stable and precise. Hence, it is suggested that the temperature-control of the temperature-adjustable biochip assembly in the present invention is dramatically steady and rapid, and accordingly the temperature-adjustable biochip assembly is sufficient to apply to manufacture of a condensed drug carrier in order to maintain the stability of manufacturing processes of the condensed drug carriers.

Through the present invention mentioned above, the temperature-adjustable biochip assembly comprises the temperature-controlling unit 2 installed on a lateral of the biochip 1, which can rapidly and stably heat up or cool down the fluids 23, 23′ via the temperature-controlling components 24, 24′ in order to precisely maintain the temperature of the fluids 23, 23′, promote the stability of temperature-control of the microfluidic channel 14, and more efficiently control the temperature of the biochip 1. As a result, it is sufficient to improve the disadvantages of the unstable heating, poor maintenance of temperature, and slow cooling of the conventional polar layer, and further provide the temperature-adjustable biochip assembly in accord with the requests of industries needed.

Although the invention has been described in detail with reference to its presently preferred embodiment, it will be understood by one of ordinary skill in the art that various modifications can be made without departing from the spirit and the scope of the invention, as set forth in the appended claims.

1. A temperature-adjustable biochip assembly, comprising:
a biochip having a first binding hole, a second binding hole, a third binding hole, and a microfluidic channel embedded inside, wherein the microfluidic channel comprises a main microfluidic channel and a sub microfluidic channel, the main microfluidic channel has a starting end and a terminal end, the sub microfluidic channel intersects the main microfluidic channel at an intermediate point between the starting and terminal ends, the first binding hole leads to the starting end of the main microfluidic channel, the second binding hole leads to the sub microfluidic channel, and the third binding hole leads to the terminal end of the main microfluidic channel; and
a temperature-controlling unit including a substance sited at a surface of the biochip, a cyclic guiding module passing through the substance, and a fluid flowed inside the cyclic guiding module, with the cyclic guiding module having a fluidic installed in the substance for guiding the fluid and a temperature-controlling component for modulating the temperature of fluid.

2. The temperature-adjustable biochip assembly as defined in claim 1, wherein the cyclic guiding module further comprises a driver to propel the circulating flow of the fluid in the fluidic.

3. The temperature-adjustable biochip assembly as defined in claim 2, wherein the cyclic guiding module further comprises a delivering channel to connect to the fluidic, the temperature-controlling component and the driver individually.

4. The temperature-adjustable biochip assembly as defined in claim 3, wherein the cyclic guiding module further comprises a fluid-controlling valve set on the delivering channel.

5. The temperature-adjustable biochip assembly as defined in claim 2, wherein the driver is a pump.

6. The temperature-adjustable biochip assembly as defined in claim 1, wherein the fluid is water or ethanol.

7. The temperature-adjustable biochip assembly as defined in claim 1, wherein the fluidic comprises a fluid tank, an inlet and an outlet, with the fluid tank communicating with the inlet and the outlet.

8. (canceled)

9. The temperature-adjustable biochip assembly as defined in claim 1, wherein the main microfluidic comprises a winding portion.

10. The temperature-adjustable biochip assembly as defined in claim 1, wherein the temperature-controlling unit is conjugated with the biochip by at least one of screw binding, thermal binding and adhesive binding.

11. The temperature-adjusting biochip assemble as defined in claim 1, wherein the biochip further comprises a first plate, a second plate, and a third plate, the microfluidic channel is arranged on the first plate, the first and second binding holes are arranged on the second plate, and the third binding hole is arranged on the third plate.
